(19) United States
(12) Patent Application Publication
(10) Pub. No.: US 2009/0232748 A1
(43) Pub. Date: Sep. 17, 2009

(54) Virucidal activities of cetylpyridinium chloride

(75) Inventor: Charles L. Capps, Little Rock, AR (US)

Correspondence Address: VINSON & ELKINS L.L.P., First City Tower, 1001 Fannin Street, Suite 2300, HOUSTON, TX, 77002-6760, US

(73) Assignee: ViraTox, L.L.C., Little Rock, AR (US)

(21) Appl. No.: 12/381,966

(22) Filed: Mar. 18, 2009

Related U.S. Application Data
(63) Continuation-in-part of application No. 10/939,307, filed on Sep. 10, 2004.
Application No. 10/939,307 is a continuation-in-part of application No. 10/703,969, filed on Nov. 7, 2003, now abandoned.

(51) Int. Cl. A61K 8/49; A01N 43/40; A01N 59/16; A01N 59/20; A61K 31/4425; A01P 1/00; A61P 31/04

(52) U.S. Cl. 424/49; 514/358; 424/641; 424/638

(57) ABSTRACT

This disclosure relates to inventive methods for inactivating viral pathogens comprising providing a virucidal composition comprising a liquid media containing less than 1% weight per volume of a quaternary ammonium compound, such as cetylpyridinium chloride and between 0% and 0.5% weight per volume of citric acid, and contacting a surface targeted for disinfection with the virucidal composition. This disclosure further relates to inventive virucidal compositions comprising a liquid media, a quaternary ammonium compound, such as cetylpyridinium chloride, at a concentration of less than 1% weight per volume, citric acid at a concentration of between 0.0% and 0.5% weight per volume, an extender, and an enhancer.

### CROSS-REFERENCE TO RELATED APPLICATIONS

This application is a continuation-in-part of U.S. patent application Ser. No. 10/939,307, filed Sep. 10, 2004, which is a continuation-in-part of U.S. patent application Ser. No. 10/703,969, filed Nov. 7, 2003, now abandoned, each of which are incorporated herein by reference in their entirety.

### THE NAMES OF PARTIES TO A JOINT RESEARCH AGREEMENT

N/A

### INCORPORATION-BY-REFERENCE OF MATERIAL SUBMITTED ON A COMPACT DISC

N/A

### BACKGROUND OF THE INVENTION

1. Field of the Invention

The present disclosure relates to the use of quaternary ammonium compounds as virucidal agents for surface contact disinfection and for the medical treatment and prevention of infection caused by viral particles. In one embodiment, the present disclosure relates to the use of cetylpyridinium chloride in a low concentration solution for contact destruction of viral particles in a relatively short time period with subsequent residual associated toxicity to viral particles. In other embodiments, the present disclosure describes the composition and application of cetylpyridinium chloride from a dry composition powder to a solution for surface disinfection; as a spray or aerosol inhalant for respiratory treatment for the destruction or inhibition of the corona virus; and as a foam, film, liquid, or suppository in the prevention of HIV transmission.

2. Description of the Related Art

Viruses are considered to be the smallest infectious agents capable of replicating in living cells. The precise mechanism of nucleic acid transfer to the host cell and the subsequent function of the host to promote production of additional progeny is not fully understood. Viruses have a core of nucleic acid surrounded by a protein coat or coats and may be furthered enveloped as in the case of the corona virus. The nucleic acids are of two types, ribonucleic acid (RNA) or deoxyribonucleic acid (DNA). This genetic material is responsible for directing replication once a suitable host is invaded. Without a protein coat, the nucleic acid is normally incapable of entering a cell.

In general, the DNA viruses multiply in the nucleus of the host cell, whereas the RNA viruses multiply in the cytoplasm. Some RNA viruses appear to emerge as buds from the cell membrane. Generally, the mixoviruses, and rhinoviruses such as the corona virus, as well as human immunodeficiency virus (“HIV”), are of the RNA types, while viruses such as the papovaviruses (herpes), the adenoviruses (pox) and the orthomyxoviridie (influenza and parainfluenza) are DNA based. Corona viruses, such as those that cause the common cold, are known to infect a wide variety of hosts. For example, corona viruses are known to infect humans, dogs, cats, pigs and birds.

Virucides play a critical role in limiting or eradicating the deleterious presence of pathogenic viruses in a wide variety of settings, such as in hospitals, patient rooms, treatment facilities, and other surfaces in which the inactivation of viruses would have beneficial effects. Nevertheless, the need for potent and effective virucides must be counterbalanced against concerns for environmental safety.

Cetylpyridinium chloride (CPC) is a quaternary ammonium compound as described in The Merck Index (11th Ed., Merck & Co., Rahway, N.J., 1989, p. 311) with antiseptic and preservative properties. It is a cationic compound that is presently known to have broad based antibacterial properties, thus making it an active ingredient for the inactivation of both gram negative and gram positive bacteria. Examples of bacteria that are known to be susceptible are: Escherichia coli, Salmonella typhimurium, Pseudomonas aeruginosa, Bacillus anthracis, Bacillus subtilis, Campylobacter, Listeria, Staphylococcus aureus, and Streptococcus pneumoniae. Cetylpyridinium chloride is also known for its antifungal properties affecting such fungi as Candida albicans and spp., Saccharomyces cerevisiae, Torulopsis glabrata, Trichophyton sp., Aspergillus flavus, Aspergillus niger, Stachybotrys atra, Chaetomium globosum, Histoplasma capsulatum, Penicillium cyclopium, and Cladosporum resinae (Reed, “Cetyl Pyridinium Chloride: Status Report.” University of Northumbria School of Applied and Molecular Biology, January 2002).

The recognized broad spectrum antimicrobial antiseptic effect of CPC without disturbance of intra-oral bacterial flora has resulted in its common use in oral rinses and lozenges. Commercial oral rinse products include, for example, Scope® (Procter and Gamble), Cepacol® (J.B. Williams), and Act® (Johnson & Johnson). Formulation concentrations of CPC in these products range in the 0.04% to 0.05% and are generally recognized as safe levels while possessing the ability to be effective against undesired microbes. There are also multiple citations of CPC efficacy as an active ingredient in the reduction and inhibition of plaque and gingivitis, thus encouraging its use as a dental hygiene constituent (Hunter-Rinderele, et al., J. Clin. Res. 72:107113, 1997). However, much evidence exists wherein the efficacy of CPC as an antimicrobial is severely hindered by commonly used ingredients in the formulations of rinses, lozenges, dentifrices and oral care products (Addy, et al., J. Dent. Res. 72:719, 1993). Other commercial antiseptic product applications include feminine washes and topical antiseptics for abrasions and minor wounds.

Elimination of viral pathogens, especially those existing upon inanimate surfaces, where such pathogens may remain active for extended periods of time, has been a long standing challenge to maintaining an antiseptic environment in a wide variety of settings. A number of disinfectants have been developed in attempts to provide an adequate mechanism for the elimination of such statically existing viral pathogens. However, many of these disinfectants tend to have less desirable characteristics. For example, some may be corrosive while others may be repugnant or cause discoloration of a treated area.

What is needed, therefore, is a virucidal agent that provides for surface contact disinfection and a composition for the medical treatment and prevention of infection caused by viral particles, while minimizing or eliminating the adverse effects that may be experienced with conventional agents.

The present disclosure is directed to overcoming, or at least reducing the effects of, one or more of the problems set forth above.

### SUMMARY OF THE INVENTION

The present disclosure includes compositions and methods for inactivating viruses residing on inanimate objects; within the respiratory tract of humans or other animals, especially the oral or nasal cavities; and with the anal or urogenital tract of humans for the prevention or inhibition of HIV. The present disclosure also includes methods and compositions for decontaminating areas, tools or devices infected by viral pathogens such as those commonly found in hospitals, public restrooms, transportation vehicles, and institutions where mass population would require sanitization of surfaces with maximum residual results.

The present disclosure includes the use of quaternary ammonium compounds, such as solubilized CPC, in a liquid or other suitable media. Certain embodiments comprise solubilizing CPC in either potable or sterile water for use as a surface contact disinfectant or as a spray in oropharyngeal applications for the destruction of viral agents in the respiratory tracts of humans or other animals. Still other embodiments of the present disclosure provide a virucidal kit comprising a quaternary ammonium containing compound, such as a CPC containing compound. In such kits, the CPC containing compound may be provided in a concentrated form for dilution to a working concentration prior to use.

Certain other embodiments of the present disclosure provide compositions that may be safely ingested by humans and other animals, thereby making the compositions and methods of the present disclosure suitable for treating humans and other animals, exposed to pathogenic viruses. In alternative embodiments, effective amounts of the quaternary ammonium compound, such as CPC, may be applied to the human and other animal being treated prior to exposure to the pathogenic viruses. Therefore, the present disclosure includes compositions and methods for both the prevention and treatment of viral infections. In these particular embodiments, the present disclosure may comprise an inactivating composition suitable for pharmaceutical administration to humans and/or other animals. Such compositions suitable for pharmaceutical administration may also comprise one or more pharmaceutically acceptable carriers. These compositions may be applied topically to mucus membranes or oral surfaces including the buccal cavity and throat as a spray or oral rinse (or other acceptable form) or to any other suitable surface of the body, including dermal surfaces and open wounds and the anal or vaginal tract, to treat or prevent viral infections.

The present disclosure provides methods for inactivating viral pathogens comprising providing a liquid virucidal composition comprising less than 1% weight per volume of a quaternary ammonium compound, such as cetylpyridinium chloride, and between 0% and 0.5% weight per volume of citric acid, and contacting the virucidal composition with a surface targeted for disinfection. In certain embodiments, the virucidal composition comprises less than 0.5% weight per volume of a quaternary ammonium compound, such as cetylpyridinium chloride, less than 0.05% weight per volume of a quaternary ammonium compound, such as cetylpyridinium chloride, less than 0.03% weight per volume a quaternary ammonium compound, such as cetylpyridinium chloride, or less than 0.025% weight per volume of a quaternary ammonium compound, such as cetylpyridinium chloride.

In certain aspects of the present disclosure, the virucidal composition further comprises at least one extender. In particular aspects, the at least one extender is a cellulose ether, including, but not limited to, hydroxypropyl methylcellulose or sodium carboxymethylcellulose. In other aspects, the at least one extender is a non-ionic detergent, including, but not limited to, Tween 80, Triton, Tyloxapol, Pluronic, or Span.

In further embodiments, the virucidal composition further comprises at least one enhancer. In certain embodiments, the enhancer is selected from a group including, but not limited to, zinc oxide, zinc acetate, zinc bacitracin, zinc carbonate, zinc citrate, zinc iodate, zinc iodide, zinc peroxide, zinc propionate, zinc stearate, zinc salicylate, cuprous iodide, and cupric oleate. In some embodiments, the virucidal composition further comprises at least one enhancer and at least one extender. In particular embodiments, the liquid is an aqueous media, such as sterile water, potable water, sterile saline, or a fatty alcohol.

In certain aspects of the disclosure, the surface targeted for disinfection is an inanimate surface. In such aspects, the virucidal composition can be is contacted with the surface targeted for disinfection through the use of, for example, an applicator, such as a wipe, a mop head, or a sprayer. In particular aspects, the virucidal composition further comprises a scented component.

In further embodiments of the present disclosure, the virucidal composition is contacted to the surface for at least 30 seconds, at least 45 seconds, at least 1 minute, at least 90 seconds, at least 2 minutes, or at least 3 minutes.

The present disclosure also provides methods for inactivating viral pathogens on inanimate surfaces comprising providing a virucidal composition comprising a liquid media containing less than 1% weight per volume of a quaternary ammonium compound, such as cetylpyridinium chloride, and between 0.0% and 0.5% weight per volume of citric acid, and contacting the virucidal composition with the targeted inanimate surface for disinfection.

The present disclosure further provides methods for the treatment of virulent infections comprising providing a pharmaceutically acceptable virucidal composition containing a quaternary ammonium compound, such as cetylpyridinium chloride, in solution in a liquid media at a concentration of less than 0.05% weight per volume and citric acid in solution in the liquid media at a concentration of between 0.0% and 0.5%, and topically applying the virucidal composition to an infected surface area of a host organism. In certain aspects, the virucidal composition comprises less than 0.03% weight per volume cetylpyridinium chloride, or less than 0.025% weight per volume cetylpyridinium chloride.

In certain embodiments, the pharmaceutically acceptable virucidal composition is contacted with the infected surface area as an oral rinse, a gel, a foam, a film, a mist, a spray, or a suppository. In other embodiments, the virucidal composition coated onto a condom. In particular embodiments, the infected surface area is a mucous membrane, including, but not limited to, a mucous in the nasal cavity, in a buccal cavity, in an oropharyngeal cavity, in a urogenital tract, in a lung, or in an anal tract. In other embodiments, pharmaceutically acceptable virucidal composition further comprises a flavoring component.

The present disclosure also provides virucidal compositions comprising a liquid media, a quaternary ammonium compound, such as cetylpyridinium chloride, in solution in a liquid media at a concentration of less than 1% weight per volume, citric acid in solution in the liquid media at a concentration of between 0.0% and 0.5%, an extender, and an enhancer. In certain embodiments, the virucidal composition is a pharmaceutically acceptable composition.

The present disclosure further provides methods for inactivating a virus in a host organism comprising providing a virucidal composition comprising a liquid media containing less than 0.05% weight per volume of a quaternary ammonium compound, such as cetylpyridinium chloride, and between 0.0% and 0.5% of citric acid, and contacting the virucidal composition with a surface contaminated with the virus.

In certain aspects, the virus is a corona virus, an orthomyxoviridie virus, or a human immunodeficiency virus. In particular aspects, the virus is orthomyxoviridie only. In other aspects, the virucidal composition is contacted with the surface contaminated with the virus in the form selected from the group consisting of an oral rinse, a gel, a foam, a film, a mist, a spray, and a suppository.

In addition, the present disclosure provides methods for inactivating virus infections, comprising providing a virucidal composition comprising a liquid media containing less than 0.05% weight per volume of a quaternary ammonium compound, such as cetylpyridinium chloride, and between 0.0% and 0.5% weight per volume of citric acid, in solution, and contacting the virucidal composition with a surface area of virally infected tissue. In particular embodiments, the surface area of virally infected tissue is in the lungs of an organism.

### DETAILED DESCRIPTION OF SPECIFIC EMBODIMENTS

As used herein, the term “inactivate” or “inactivating” means having the ability to destroy, eliminate or reduce the capacity of a viral pathogen to infect and/or cause a pathological response in a host; wherein such inactivating ability preferably provides at least a one-log order reduction (90%) in the ability of the viral pathogens to infect and/or cause a pathological response in a host, more preferably a two-log order reduction (99%), and most preferably a three-log order reduction (99.9%).

As used herein, the term “contact” or “contacted” refers to bringing one or more of the compositions of the present disclosure into contact with a viral pathogen such that the composition(s) of the present disclosure may inactivate the viral pathogens.

As used herein, the term “enhancers” describes compounds or substances that act to enhance or accelerate the ability of a quaternary ammonium compound, such as CPC, to inactivate viral pathogens.

As used herein, the term “extenders” describes compounds or substances that act to extend or prolong the inactivating ability of a quaternary ammonium compound, such as CPC, on viral pathogens.

As used herein, the term “pharmaceutically acceptable” refers to compositions that do not produce significant adverse reactions when administered or applied to a particular host (e.g., a human or other animal).

As used herein, the term “pharmaceutically acceptable carrier” includes any and all solvents, media, coating or wetting agents, and the like, as well as certain enhancers and extenders, which when utilized remain pharmaceutically acceptable.

Illustrative embodiments of the disclosure are described below. In the interest of clarity, not all features of an actual implementation are described in this specification. It will of course be appreciated that in the development of any such actual embodiment, numerous implementation-specific decisions must be made to achieve the developer's specific goals, such as compliance with system-related and business-related constraints, which will vary from one implementation to another. Moreover, it will be appreciated that such a development effort might be complex and time-consuming, but would nevertheless be a routine undertaking for those of ordinary skill in the art having the benefit of this disclosure.

The present disclosure provides for compositions of a solubilized quaternary ammonium compound, such as CPC, in a liquid or other suitable media and the use of these compositions to inactive viral pathogens. The present disclosure further comprises solubilizing a quaternary ammonium compound, such as CPC, in any suitable liquid media, such as potable or sterile water or an isotonic saline solution, for uses as a surface contact disinfectant or as a spray in applications for the destruction of viral agents in the respiratory tracts of humans or other animals. Solubilizing a quaternary ammonium compound, such as CPC, should be done by adding selected amounts of the quaternary ammonium compound, such as CPC powder, into the liquid media of choice. Alternative embodiments of the compositions of the present disclosure also include the addition of a stabilizing agent, such as polypropylene glycol, to stabilize and reduce the freezing point of the resulting solution.

Quaternary Ammonium Compounds

The presently disclosed compositions comprise at least a first quaternary ammonium compound. In certain embodiments, the quaternary ammonium compound is: CPC, N-alkyldimethyl benzyl ammonium saccharinate; 1,3₅5-triazine-1,3,5(2H,4H,6H)-triethanol; 1-decanaminium, N-decyl-N,N-dimethyl-chloride, didecyl dimethyl ammonium chloride; 2-(2-(p-(diisobuyl)cresosxy)ethoxy) ethyl dimethyl benzyl ammonium chloride; 2-(2-(p-(diisobutyl)phenoxy)ethoxy) ethyl dimethyl benzyl ammonium chloride; alkyl 1 or 3 benzyl-1-(2-hydroxethyl)-2-imidazolinium chloride; alkyl bis(2-hydroxyethyl) benzyl ammonium chloride; alkyl demethyl benzyl ammonium chloride; alkyl dimethyl 3,4-dichlorobenzyl ammonium chloride (100% C12); alkyl dimethyl 3,4-dichlorobenzyl ammonium chloride (50% C14, 40% C12, 10% C16); alkyl dimethyl 3,4-dichlorobenzyl ammonium chloride (55% C14, 23% C12, 20% C16); alkyl dimethyl benzyl ammonium chloride; alkyl dimethyl benzyl ammonium chloride (100% C14); alkyl dimethyl benzyl ammonium chloride (100% C16); alkyl dimethyl benzyl ammonium chloride (41% C14, 28% C12); alkyl dimethyl benzyl ammonium chloride (47% C12, 18% C14); alkyl dimethyl benzyl ammonium chloride (55% C16, 20% C14); alkyl dimethyl benzyl ammonium chloride (58% C14, 28% C16); alkyl dimethyl benzyl ammonium chloride (60% C14, 25% C12); alkyl dimethyl benzyl ammonium chloride (61% C11, 23% C14); alkyl dimethyl benzyl ammonium chloride (61% C12, 23% C14); alkyl dimethyl benzyl ammonium chloride (65% C12, 25% C14); alkyl dimethyl benzyl ammonium chloride (67% C12, 24% C14); alkyl dimethyl benzyl ammonium chloride (67% C12, 25% C14); alkyl dimethyl benzyl ammonium chloride (90% C14, 5% C12); alkyl dimethyl benzyl ammonium chloride (93% C14, 4% C12); alkyl dimethyl benzyl ammonium chloride (95% C16, 5% C18); alkyl dimethyl benzyl ammonium chloride (and) didecyl dimethyl ammonium chloride; alkyl dimethyl benzyl ammonium chloride (as in fatty acids); alkyl dimethyl benzyl ammonium chloride (C12-C16); alkyl dimethyl benzyl ammonium chloride (C12-C18); alkyl dimethyl benzyl and dialkyl dimethyl ammonium chloride; alkyl dimethyl dimethybenzyl ammonium chloride; alkyl dimethyl ethyl ammonium bromide (90% C14, 5% C16, 5% C12); alkyl dimethyl ethyl ammonium bromide (mixed alkyl and alkenyl groups as in the fatty acids of soybean oil); alkyl dimethyl ethylbenzyl ammonium chloride; alkyl dimethyl ethylbenzyl ammonium chloride (60% C14); alkyl dimethyl isoproylbenzyl ammonium chloride (50% C12, 30% C14, 17% C16, 3% C18); alkyl trimethyl ammonium chloride (58% C18, 40% C16, 1% C14, 1% C12); alkyl trimethyl ammonium chloride (90% C18, 10% C16); alkyldunethyl (ethylbenzyl) ammonium chloride (C12-18); di-(C8-10)-alkyl dimethyl ammonium chlorides; dialkyl dimethyl ammonium chloride; dialkyl dimethyl ammonium chloride; dialkyl dimethyl ammonium chloride; dialkyl methyl benzyl ammonium chloride; didecyl dimethyl ammonium chloride; diisodecyl dimethyl ammonium chloride; dioctyl dimethyl ammonium chloride; dodecyl bis(2-hydroxyethyl)ocryl hydrogen ammonium chloride; dodecyl dimethyl benzyl ammonium, chloride; dodecylcarbamoyl methyl dimethyl benzyl ammonium chloride; heptadecyl hydroxyethylimidazolinium chloride; hexahydro-1,3,5-tris(2-hydroxyethyl)-s-triazine; myristalkonium chloride (and) Quat RNIUM 14; N,N-dimethyl-2-hydroxypropylammonium chloride polymer; n-alkyl dimethyl benzyl ammonium chloride; n-alkyl dimethyl ethylbenzyl ammonium chloride; n-tetradecyl dimethyl benzyl ammonium chloride monohydrate; octyl decyl dimethyl ammonium chloride; octyl dodecyl dimethyl ammonium chloride; octyphenoxyethoxyethyl dimethyl benzyl ammonium chloride; oxydiethylenebis (alkyl dimethyl ammonium chloride); quaternary ammonium compounds, dicoco alkyldimethyl, chloride; trimethoxysily propyl dimethyl octadecyl ammonium chloride; trimethoxysilyl quats, and trimethyl dodecylbenzyl ammonium chloride, or any combination thereof.

The compositions of the present disclosure can also include a dual cationic component. The dual cationic component of the present disclosure includes embodiments wherein the quaternary ammonium compound is selected from the listing in the previous paragraph. In certain embodiments, the dual cationic component is Maquat™ 2525 M 50% (Mason Chemical Company, Arlington Heights, Ill.; hereinafter “Maquat”) at a concentration ranging from greater than 0.0% to 5% weight per weight. Maquat is comprised of two quaternary ammonium compounds in equal weight to total composition of matter. The two cationic surfactants in Maquat are alkyl dimethyl benzyl ammonium chloride and alkyl dimethyl ethyl benzyl ammonium chloride. Both of these cationic surfactants are known to possess limited antimicrobial effects and are classified as disinfectants.

Surface Contact Disinfectant

In embodiments of the present disclosure that are directed toward surface contact disinfectants and disinfection, the quaternary ammonium compound solution, such as a CPC solution, generally may range in concentration from greater than 0.0% up to around 1.0%, the concentrations varying as to specific use. In certain embodiments directed toward household applications, a solution concentration of greater than 0.0% to 1.0%, preferably from 0.01% to 0.5%, more preferably from 0.02% to 0.05%, most preferably around 0.025%, would provide a benign but effective antibacterial disinfectant that would additionally promote the inactivation of certain viral agents, including the corona virus and the influenza virus and the HIV virus. In applications requiring more aggressive disinfection where more virulent strains of viral organisms exist, higher concentrations of CPC up to around 1.0% may be required to assure maximal contact destruction of contaminants on surfaces with residual presence to inhibit further contamination within a reasonable period of time.

Certain quaternary ammonium compounds, such as CPC, also possess a surfactant property and aggressively adhere to solid surfaces thus promoting a residual action. Extenders, such as cellulose ethers, including, but not limited to, hydroxypropyl methylcellulose or sodium carboxymethylcellulose, may be used in various concentrations as adjuncts in certain embodiments of the present disclosure for extending or enhancing the toxicity and longevity of residual inactivating effect of the quaternary ammonium compound. These compounds are widely recognized as viscosity modifiers, emulsifiers, protective colloids in emulsions, and as film forming agents. Viscosity properties are dependent on the degree of substitution as a water soluble polymer. For example, in the case of hydroxypropyl methylcellulose, a substitution of 0.01% to 2.5% will cause film-forming and encapsulation, while 0.5% to 5.0% of carboxymethylcellulose will accomplish similar results. In certain embodiments of the present disclosure these extender compounds may increase surface attachment and upon drying may result in a solid film with varying degrees of water resistance. In certain other embodiments, it is contemplated that the extender is a non-ionic detergent. Non-ionic detergents that find use in the present disclosure include, but are not limited to, varying concentrations, which would be readily ascertainably to one of skill in the art, of Tween 80, Triton, Tyloxapol, Pluronic and Span.

Other embodiments of the present disclosure may comprise the addition of enhancers, which function to accelerate the inactivating properties of CPC. Alternative embodiments of the compositions of the present disclosure may comprise solutions containing zinc or copper compounds, which include, but are not limited to, zinc oxide, zinc acetate, zinc bacitracin, zinc carbonate, zinc citrate, zinc iodate, zinc iodide, zinc peroxide, zinc propionate, zinc stearate, zinc salicylate, cuprous iodide, and cupric oleate. Still other embodiments of the present disclosure may contain citric acid to act as an enhancer or accelerator. It is believed that the addition of the COOH molecules in the dicarboxylic acid increases the reactivity of the long organic tail of the quaternary ammonium compound. The use of citric acid as an enhancer is of particular efficacy in uses directed toward orthomyxoviridie. In fact, of the coronavirus, RSV virus, orthomyxoviridie, and the retrovirus, the orthomyxoviridie (influenza and parainfluenza) in particular require the citric acid addition to be most effective.

The concentrations necessary will vary from enhancer to enhancer but would be readily ascertainable to one of skill in the art. For example zinc oxide may be used as an enhancer at concentrations of greater than 0.0% weight per volume to as high as around 2.5% weight per volume, while citric acid may be used at concentrations ranging from greater than 0.0% weight per volume to as high as around 0.5% weight per volume.

The compositions and methods of the present disclosure also contemplate combining the quaternary ammonium compound compositions with various cleansing applicators, such as wipes, mop head, sprays, and the like.

The methods of the present disclosure comprise contacting quaternary ammonium compound containing compositions for a time sufficient to inactivate the pathogenic agent or to inhibit the growth of the agent. Contact times sufficient for the inactivation of pathogenic agents may vary from agent to agent but will be readily ascertainable to one of skill in the art. Time periods will preferably range from greater than 0 seconds to around 3 minutes, more preferably from 30 seconds to 1 minute, most preferably a sufficient time period would be around 45 seconds. In other embodiments, the present disclosure provides a method of decontaminating an environmental surface harboring harmful or undesired pathogens. In one such embodiment, the pathogenic agent is associated with an environmental surface and the method comprises contacting the environmental surface with an amount of the composition sufficient for decontaminating the surface. While it may be so desired, decontamination need not result in total elimination of the pathogen, preferably decontamination will result in a least a one-log reduction (90%) in the presence of active viral pathogens, more preferably a two-log reduction (99%), and even more preferably a three-log reduction (99.9%). In some embodiments, the compositions and methods further comprise dyes, paints, and other marking and identification compounds as to ensure that a treated surface has been sufficiently treated with the compositions of the present disclosure.

The present disclosure further provides methods for protecting (e.g., protecting from contamination of a microorganism) or decontaminating an area (e.g., decontaminating an area by inactivating viral particles in the area) comprising exposing the area to a quaternary ammonium compound containing composition comprising an extender and/or enhancer. The exposure times for such areas may vary from area to area and from extender and/or enhancer to extender and/or enhancer but will be readily ascertainably one of skill in the art. Such exposure times will preferably range from greater than 0 seconds to around 3 minutes, more preferably from 30 seconds to 1 minute, most preferably a sufficient time period would be around 45 seconds. These methods may be applied to any type of area or item (e.g., a medical device or treatment table or paper/cloth diapers or mats). For example, in some embodiments, the area may be any variety of hard or soft surfaces, whether porous or non-porous.

Other embodiments of the present disclosure provide a virucidal kit comprising a quaternary ammonium compound containing composition. In such kits, the quaternary ammonium compound containing composition may be provided in a concentrated form for dilution to a working concentration prior to use. These kits may also contain one or more extenders and or enhancers. Furthermore, these kits may provide instructions as to a method of use and or desired dilution ratios to be employed. In some embodiments such kits may provide methodologies for targeting particular types of viral pathogens, which may comprise variations in quaternary ammonium compound concentrations, or the use of particular extenders and/or enhancers.

Pharmaceutical Applications

The present disclosure also comprises compositions and methods that are useful in the treatment and prevention of virally mediated pathogenic responses in humans and other animals. Viruses mediating such responses would preferably be viruses infecting the respiratory tract of humans and other animals, especially the upper respiratory tract. For example, suitable quaternary ammonium compound containing compositions are effective in inactivating the corona virus, which is capable of infecting a wide variety of hosts including humans, dogs, cats, pigs and birds. Quaternary ammonium compound containing compositions are also capable of inactivating orthomyxoviridie or influenza viruses. The compositions and methods of the present disclosure will also be useful in the treatment and prevention of virally mediated pathogenic responses on dermal surfaces and open wounds in humans and other animals. For example, in the treatment and prevention of pathogenic states that result from dermal contact with anthrax. Furthermore, the quaternary ammonium compound containing compositions can inactivate HIV. Therefore, in certain embodiments, the present disclosure may be useful in the treatment and prevention of sexually transmitted diseases, such as HIV, via application to the anal or urogenital tracts in the form of a foam, film, liquid or suppository.

In some embodiments, the present disclosure provides compositions and methods suitable for treating humans and other animals exposed to pathogen viruses. In alternative embodiments, effective amounts of the quaternary ammonium compound are applied to the human and other animal being treated prior to exposure to the viral pathogens. Therefore, the present disclosure comprises compositions and methods for both the prevention and treatment of viral infections. In these particular embodiments, the present disclosure may comprise an inactivating composition suitable for pharmaceutical administration to humans and/or other animals.

In the case of upper respiratory tract infections, such as the flu or the common cold, the viral infection remains primarily located in the throat and respiratory tract and causes a susceptibility to infection by other microbial organisms by weakening tissues and inviting secondary bacterial infections. In the present disclosure, delivery of a quaternary ammonium compound to these areas via a spray, such as by a pump device or a propelled aerosol, would enable contact destruction of the virus as well as secondary infectious bacterial agents sensitive to the quaternary ammonium compound. Such devices are readily available in the marketplace and include metered dose inhalants by Cambridge Consultants (Boston, Mass.) or Wilden Pump and Engineering, LLC (Grand Terrace, Calif.). Pharmaceutically acceptable quaternary ammonium compound containing compositions may be utilized in methods of the present disclosure to directly target tissue areas of infection within the host. For example, an oropharyngeal spray or rinse may by used to deliver a suitable quaternary ammonium compound containing composition to the throat, buccal cavity and/or nasal passages of an infected individual in concentrations that would be effective in inactivating the viral pathogens. Furthermore, such a spray or rinse may even be used to inactive viral pathogens upon suspected exposure prior to actual infection.

In the case of an oropharyngeal application, a quaternary ammonium compound, such as CPC, in the range of greater than 0.0% to 0.5%, preferably 0.01% to 0.1%, more preferably 0.025% to 0.05%, in sterile solution would be applied as a spray to the area of the buccal cavity and throat to enable contact destruction of viral agents of the mucosal surfaces to reduce or eliminate infection of viral agents. The compositions and methods of the present disclosure are also useful in the treatment and prevention of virally-mediated pathogenic responses in the lungs of humans and other animals. In the case of the treatment and prevention of viral pathogens in the lungs of humans and other animals, quaternary ammonium compound containing pharmaceutically acceptable compositions may be applied as a mist or mist-like inhalant for direct contact with the tissues of the bronchial tract and lung alveoli and surrounding tissue. In such intra-lung applications, useful quaternary ammonium compound concentrations in sterile solution may be in the range of greater than 0.0% to 0.5% weight per volume, preferably 0.01% to 0.1%, more preferably 0.025% to 0.05%. Additional affectation of viral infection may occur as the transport of the quaternary ammonium compound solution ensues within cellular structures of tissue hosting viral agents. Since quaternary ammonium compounds are highly cationic, they are readily adsorbed and retained on oral and mucosal surfaces, providing a mechanism for extended residual action.

Such compositions suitable for pharmaceutical administration may also comprise one or more pharmaceutically acceptable carriers. These compositions can be applied topically to mucous membranes or oral surfaces including the buccal cavity and throat as a spray or oral rinse (or other acceptable form) or to any other suitable surface of the body, including dermal surfaces and open wounds, to treat or prevent viral infections.

Certain embodiments of the present disclosure also provide compositions and methods for the treatment and prevention of sexually transmitted diseases, particularly HIV. The quaternary ammonium compound containing compounds may be provided in the form of a gel, foam, film, liquid or suppository. In such forms, the compounds may be applied directly to the anal or urogenital tracts of an infected individual. Alternatively, the compounds of the present disclosure may be useful in indirect applications such as an additive in the packaging of condoms and other non-pharmaceutical birth control devices.

Extenders may be used in compositions of the present disclosure in addition to or as pharmaceutically acceptable carries. Such extenders include, but are not limited to, the cellulose ethers hydroxypropyl methylcellulose or sodium carboxymethylcellulose, which may be used in various concentrations. In certain other embodiments, it is contemplated that the extender is a non-ionic detergent. Non-ionic detergents that find use in the present disclosure include, but are not limited to, varying concentrations, which would be readily ascertainably to one of skill in the art, of Tween 80, Triton, Tyloxapol, Pluronic and Span.

Alternate embodiments of the present disclosure may contain suitable enhancers to accelerate the inactivating properties of the quaternary ammonium compound. For example, zinc, in the form of zinc oxide, may be included in the quaternary ammonium compound containing compositions to accelerate the quaternary ammonium compound mediated inactivation of viral particles. In certain other embodiments, the composition may contain citric acid to act as an enhancer or accelerator. The use of citric acid as an enhancer is of particular efficacy in uses directed toward orthomyxoviridie. In still other embodiments of the present disclosure quaternary ammonium compound containing compositions may contain both extenders and enhancers.

The concentrations necessary will vary from enhancer to enhancer but would be readily ascertainable to one of skill in the art. For example zinc oxide may be used as an enhancer at concentrations of greater than 0.0% weight per volume to as high as around 2.5% weight per volume, while citric acid may be used at concentrations ranging from greater than 0.0% weight per volume to as high as around 0.5% weight per volume.

Additionally, in still other embodiments of the present disclosure, the formulations further comprise any number or variation of coloring or flavoring agents (e.g., dyes and peppermint oil).

While the invention has been described with particular reference to specific embodiments thereof, it is to be understood that the forms of the invention shown and described in detail are to be taken as preferred embodiments thereof. Various changes and modifications may be resorted to without departing from the spirit and scope of the invention as defined by the claims.

### EXAMPLES

The present disclosure derives from in vivo trials and challenge studies (1) in determining whether CPC may be effective as an antiviral agent and, if effective, the minimum inhibitory dosage; (2) in determining any adverse effects of the host cells and dosages thereto; and, (3) in comparing antiviral effects of CPC, benzalkonium chloride, and triclosan. As an exemplary embodiment, the present disclosure entails a method to directly measure the extent to which a potential antiviral substance inhibits the effects of viral infection in a tissue culture. Plaque reduction is recommended for virus and cell line combinations that produce a well-defined plaque. Other cytopathic effects (“CPE”) can be measured in those virus and cell lines that do not produce well-defined plaques, or where a specific cytopathic effect may be a defining characteristic.

### Example 1

### Corona Virus

Feline Infectious Peritonitis Type 2 (“FIP Type 2”) corona virus cultures were grown in CrFK cell culture in order to demonstrate plaque formation and the effect of CPC compounds of the present disclosure on corona virus activity. Untreated virus served as controls. Alternatively, virus suspensions were exposed to three concentrations of CPC for 300 seconds. The concentrations were produced by adding certified USP grade cetylpyridinium chloride powder to sterile water to produce 10 ml each of solution concentrations at 0.025%, 0.05%, and 0.10%. At the end of the exposure, the virus was titrated in CrFK cell culture. Viral titration is accomplished at each test article concentration by seeding 0.1 ml of a serial dilution of treated virus to the CrFK cell culture. Serial dilutions are prepared from 10⁻¹ to 10⁻⁸ of the original virus concentration, or five million/ml (6.7 log units) and each dilution then placed in each of four wells (replicates) of a 96 well microplate. The inoculated cells (80 μl per well) were then incubated at 37° C. in a CO₂ incubator for five days. They were then observed microscopically for CPE inhibition, reporting titers as TCID 50/ml, also expressed as plaque forming units (pfu's). The corrected virus titer (# positives×dilution) is reported in log/ml. Virucidal activity is thus defined as the number of logs reduction of virus titer between the test article and the control (Reed and Muench, Am. J. Hyg. 27:493-497, 1938).

Table 1 shows the results of the bioassay of FIP-2 corona virus against CPC, and demonstrates the efficacy of CPC in the destruction of FIP Type 2 corona virus.

TABLE 1

| | | | |
|---|---|---|---|
| CPC % | FIP-2 Titer | Reduction in Titer | % Reduction |

| | | | | | |
|---|---|---|---|---|---|
| 0.10 | <3.50 | logs/ml * | >3.20 | logs | 99.97 |
| 0.05 | <3.50 | logs/ml * | >3.20 | logs | 99.97 |
| 0.025% | 3.30 | logs/ml | 3.40 | logs | 99.95 |

| | | | | |
|---|---|---|---|---|
| Control | 6.70 | logs/ml | N/A | N/A |

* Dilutions of 10 and below at CPC concentrations of 0.10% and 0.05% did demonstrate cell toxicity and could not be read with certainty. No virus were observed.
Note:
6.7 log units = 5,000,000 virus particles; 3.30 log units = ~2,100 virus particles.

In the present disclosure, a significant reduction in virus titer occurred as a result of a five minute exposure of a viral suspension containing five million virus/ml in the presence of a 0.025% concentration of CPC. This reduction transposes into a reduction of greater than 99.95% of viral particles present. Assuming the same killing effect on a surface area, the application of CPC at a concentration of 0.025%/in² would result in a greater than 3-log reduction of virus. Comparative testing with benzalkonium chloride and triclosan as substitute active ingredients in the three concentrations as employed with CPC produced disparate results and demonstrated increased cellular toxicity. With benzalkonium chloride, only the 0.10% exposure resulted in viral reduction of which a percentage reduction was approximately 20%. Triclosan demonstrated cellular toxicity at all three levels with no viral reduction at all three levels. In the present disclosure, CPC at a concentration of 0.025% in solution consistently demonstrated a greater than 3-log lethal toxicity of the FIP Type 2 corona virus for five minutes without adverse cytopathic effects.

### Example 2

### Orthomyxoviridie

Orthomyxoviridie virus cultures were grown in MDCK (NBL-2)5 (ATCC type CCL-34) cell culture in order to demonstrate plaque formation and the effect of CPC compounds of the present disclosure on influenza type viral activity. Influenza virus, specimen influenza A/Equi 2?Miami 1/63, ATCC VR517, was passaged in cell culture to provide viral particles sufficient for conducting the in vitro assays. Serial dilutions were prepared from 10⁻¹ to 10⁻⁸ of the original virus concentration, or five million/ml (6.7 log units) and each dilution then placed in each of four wells (replicates) of a 96 well microplate. Control titers averaged 6.5 logs/ml. Virus suspensions were exposed to three concentrations of CPC for 300 seconds. The inoculated cells (80 μl per well) were then incubated at 37° C. in a CO₂ incubator for five days. Untreated virus served as a base line control.

Upon introduction of 0.05% and 0.025% of CPC, no significant reduction was observed in the titer after a five minute exposure. In addition, subsequent tests involving treatment with TSP and acetic acid at 0.1% addition to the CPC for purposes of pH adjustment showed no significant impact on the virus titer.

The addition of 0.1% citric acid (weight/volume) to both the 0.025% (weight/volume) and 0.05% (weight/volume) CPC solutions, however, resulted in a greater than 3.5 log reduction to the virus titers. Furthermore, the use of test solutions containing 0.025% CPC and 0.1% citric acid caused no observed cytotoxic affects. Additional testing in the presence of sera also resulted in the same log reduction. Furthermore, the pH of both the citric and acetic acid at 0.1% was 2.3 and neither solution was cytotoxic at those levels. All results were based upon the Reed and Muench method.

### Example 3

### FeLV

FeLV retrovirus cultures were grown in CrFK (ATCC type CCL-94) cell culture in order to demonstrate plaque formation and the effect of CPC compounds of the present disclosure on the feline surrogate to HIV. FeLV retrovirus was gown in cell culture to a titer of 6.0 logs/ml. Initial testing for cytotoxicity resulted in data consistent with the corona virus testing.

Briefly, virus suspensions were exposed to three concentrations of CPC, 0.025%, 0.05%, and 0.10%, for 300 seconds. Untreated virus served as controls. At the end of the exposure, the virus was titrated in CrFK cell culture. Viral titration was accomplished at each test article concentration by seeding 0.1 ml of a serial dilution of treated virus to the CrFK cell culture. Serial dilutions were prepared from 10⁻¹ to 10⁻⁸ of the original virus concentration, or five million/ml (6.7 log units) and each dilution then placed in each of four wells (replicates) of a 96 well microplate. The inoculated cells (80 μl per well) are then incubated at 37° C. in a CO₂ incubator for five days. CPC treatment at a concentration of 0.025% was not cytotoxic, while concentrations of 0.05% and 0.1% were.

CPC at a CPC level of 0.025%, both in conjunction 0.1% citric acid and without it, caused a greater than 3.5 logs/ml reduction in the viral titer. The presence or absence of sera had no effect on the CPC mediated inactivation of the FeLV virus. Furthermore, exposure of the virus to all three levels of CPC with or without citric acid at 0.1% resulted in a 100% reduction of the FeLV virus. All results were based upon the Reed and Muench method.

### Example Summary

It would be recognized by those in the art that the FIP-2 corona and orthomyxoviridie viruses represent an enduring and resilient viral specimen with high resistance to antibiotics and biocides. Corona is an enveloped virus, closely resembling the SARS virus, and is a surrogate for laboratory testing of the SARS virus. The corona and orthomyxoviridie viruses are hardy organisms and have an ability to reside outside of host systems for extended periods of time. These viruses have a resistance to drying, according to types, and may reside on surfaces for extended periods with full ability to transfer to a host and cause infection.

Based upon the studies of the present disclosure, application of a 0.025% CPC solution to solid surfaces via a spray device or wipe or direct liquid pour would result in an effective disinfectant application and eliminate the corona and orthomyxoviridie viruses. Additionally, since the studies substantiate no adverse cellular affectation at the 0.025% solution level, the introduction of the certain compositions of the present disclosure to the buccal cavity and throat area in humans may provide a benign but effective antiseptic and inhibitor of the corona and orthomyxoviridie viruses and other less hardy viruses. In addition, as the prior art will further support the antibiotic and antifungal properties associated with CPC, it would provide another benefit in the use of the compound for treatment of viral maladies such as the common cold, flu, or SARS. Specifically, the addition of a 0.025% CPC solution to mucosal surfaces of the respiratory tract would benefit the infected individual by inhibiting secondary infections associated with bacteria.

The particular embodiments disclosed above are illustrative only, as the invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular embodiments disclosed above may be altered or modified and all such variations are considered within the scope and spirit of the invention. Accordingly, the protection sought herein is as set forth in the claims below.

What is claimed:

1. A method for inactivating viral pathogens comprising:
(a) providing a liquid virucidal composition comprising:
(i) less than 1% weight per volume of cetylpyridinium chloride; and
(ii) between 0% and 0.5% weight per volume of citric acid; and
(b) contacting the virucidal composition with a surface targeted for disinfection.

2. The method of claim 1, wherein the virucidal composition comprises less than 0.03% weight per volume cetylpyridinium chloride.

3. The method of claim 1, wherein the virucidal composition further comprises at least one extender.

4. The method of claim 3, wherein the at least one extender is a cellulose ether or a non-ionic detergent.

5. The method of claim 1, wherein the virucidal composition further comprises at least one enhancer.

6. The method of claim 5, wherein the at least one enhancer is zinc oxide, zinc acetate, zinc bacitracin, zinc carbonate, zinc citrate, zinc iodate, zinc iodide, zinc peroxide, zinc propionate, zinc stearate, zinc salicylate, cuprous iodide, or cupric oleate.

7. The method of claim 1, wherein the virucidal composition further comprises at least one enhancer and at least one extender.

8. The method of claim 1, wherein the liquid is sterile water, potable water, sterile saline, or a fatty alcohol.

9. The method of claim 1, wherein the surface targeted for disinfection is an inanimate surface.

10. The method of claim 9, wherein the virucidal composition is contacted with the surface targeted for disinfection through the use of an applicator.

11. The method of claim 10, wherein the applicator is a wipe, a mop head or a sprayer.

12. The method of claim 9, wherein the virucidal composition further comprises a scented component.

13. The method of claim 1, wherein the surface targeted for disinfection is a mucous membrane.

14. The method of claim 13, wherein the mucous membrane is in a nasal cavity, a buccal cavity, an oropharyngeal cavity, a lung, a urogenital tract, or an anal tract.

15. The method of claim 13, wherein the virucidal composition is contacted with the surface targeted for disinfection as an oral rinse, a gel, a foam, a film, a mist, a spray or a suppository.

16. The method of claim 13, wherein the virucidal composition further comprises a flavoring component.

17. The method of claim 1, wherein the virucidal composition is contacted to the surface targeted for disinfection for at least 45 seconds.

18. The method of claim 17, wherein the virucidal composition is contacted to the surface targeted for disinfection for at least 3 minutes.

19. A method for inactivating a virus in a host organism comprising:
(a) providing a liquid virucidal composition comprising:
(i) less than 0.05% weight per volume of cetylpyridinium chloride; and
(ii) between 0% and 0.5% weight per volume of citric acid; and
(b) contacting the host organism with the virucidal composition.

20. The method of claim 19, wherein the virucidal composition further comprises less than 0.025% weight per volume of cetylpyridinium chloride.

21. The method of claim 19, wherein the virucidal composition is a pharmaceutically acceptable composition.

22. The method of claim 19, wherein the virucidal composition further comprises at least one extender, at least one enhancer, or at least one enhancer and at least one extender.

23. The method of claim 19, wherein the virucidal composition is an oral rinse, a gel, a foam, a film, a mist, a spray, or a suppository.

24. The method of claim 19, wherein the virus is a corona virus, an orthomyxoviridie virus, or a human immunodeficiency virus.

25. The method of claim 19, wherein a mucous membrane of the host organism is contacted with the virucidal composition.

26. The method of claim 19, wherein the host organism is contacted with a condom coated with the virucidal composition.

27. A virucidal composition comprising:
(a) a liquid media;
(b) cetylpyridinium chloride at a concentration of less than 1% weight per volume;
(c) citric acid at a concentration of between 0% and 0.5% weight per volume;
(d) an extender; and
(e) an enhancer.

28. The composition of claim 27, wherein the composition is pharmaceutically acceptable.
